(19) United States
(12) Patent Application Publication
(10) Pub. No.: US 2014/0288445 A1
(43) Pub. Date: Sep. 25, 2014

(54) BLOOD PRESSURE MONITORING METHOD

(71) Applicant: MURATA MANUFACTURING CO., LTD., Nagaokakyo-shi (JP)

(72) Inventors: Joonas MAKKONEN, Espoo (FI); Ulf MERIHEINÄ, Soderkulla (FI); Pekka KOSTIAINEN, Helsinki (FI); Antti FINNE, Vantaa (FI)

(73) Assignee: MURATA MANUFACTURING CO., LTD., Nagaokakyo-shi (JP)

(21) Appl. No.: 14/219,235

(22) Filed: Mar. 19, 2014

(30) Foreign Application Priority Data
Mar. 22, 2013 (FI) 20135274
Dec. 20, 2013 (FI) 20136306

(51) Int. Cl. A61B 5/022

(52) U.S. Cl. CPC A61B 5/022
USPC 600/490

(57) ABSTRACT

A device, system and method for monitoring blood pressure information of a user. A device is configured with at least one pressure sensor, a fastening element, and a processing component. In the method the pressure sensor is detachably attached to a position on the outer surface of a tissue of the user. The pressure sensor generates signal that vary according to deformations of the tissue in response to an arterial pressure wave expanding or contracting a blood vessel underlying the tissue. The signal is used to compute pulse wave parameters representing detected characteristics of the progressing arterial pressure wave of the user and blood pressure value of the user.

### FIELD OF THE INVENTION

The present invention relates to monitoring vital signs of a user and especially to a device, system, method and a computer program product for monitoring blood pressure information of a user according to the preambles of the independent claims.

### BACKGROUND OF THE INVENTION

Statistics of World Health Organization report that in 2002 cardiovascular diseases represented approximately one third of all reported deaths in non-communicable diseases globally. These diseases are considered a severe and shared risk, and a majority of the burden is in low- and middle-income countries. One factor that increases the risk of heart failures or strokes, speeds up hardening of blood vessels and reduces life expectancy is Hypertension, HTN (also called as High Blood Pressure, HBP).

Hypertension is a chronic health condition in which the pressure exerted by circulating blood upon the walls of blood vessels is elevated. In order to ensure appropriate circulation of blood in blood vessels, the heart of a hypertensive person must work harder than normal, which increases the risk of heart attack, stroke and cardiac failure. However, healthy diet and exercising can significantly improve blood pressure control and decrease the risk of complications, also efficient drug treatments are also available. It is therefore important to find persons with elevated blood pressures and monitor their blood pressure information on a regular basis.

During each heartbeat, the blood pressure varies between a maximum (systolic) and a minimum (diastolic) pressure. A traditional non-invasive way to measure blood pressure has been to use a pressurized cuff and detect the pressure levels where the blood flow starts to pulsate (cuff pressure exceeds diastolic pressure) and where there is no flow at all (cuff pressure exceeds systolic pressure). However, it has been seen that users tend to consider the measurement situations, as well as the pressurized cuff tedious and even stressing, especially in long-term monitoring. Also the well-known white-coat syndrome tends to elevate the blood pressure during the measurement, and lead to inaccurate diagnoses.

Patent publication U.S. Pat. No. 6,533,729 discloses a blood pressure sensor that includes a source of photo-radiation, an array of photo-detectors, and a reflective surface that is placed adjacent to the location where the blood pressure data is to be acquired. Blood pressure fluctuations translate to deflections of the patient's skin and these deflections show as scattering patterns detected by the photo-detectors. The solution relieves users of cuffs and compressors, but it requires a relatively complicated calibration procedure using known blood pressure data and scattering patterns, which are obtained while the known blood pressure is obtained at a known hold down pressure. During data acquisition, scattering patterns are linearly scaled to the calibrated values of signal output and hold down pressure.

Patent application publication US2005/0228299 discloses a patch sensor for measuring blood pressure without a cuff. Also this solution requires a separate calibration process that applies a conventional blood pressure cuff to generate a calibration table to be used in subsequent measurements.

### SUMMARY

The object of the present invention is to provide an improved non-invasive blood pressure information monitoring solution where at least one of the disadvantages of the prior art are eliminated or at least alleviated. The objects of the present invention are achieved with a device, system, method and a computer program product according to the characterizing portions of the independent claims.

The preferred embodiments of the invention are disclosed in the dependent claims.

The present invention is based on measuring and analysing a pulse wave for estimating diastolic and systolic blood pressure. The configuration is unnoticeable; still it provides very accurate results.

According to one embodiment a device is presented, comprising at least one pressure sensor. The device comprises a fastening element for detachably attaching the pressure sensor to a position on the outer surface of a tissue of a user. The pressure sensor is configured to generate a signal that varies according to deformations of the tissue in response to an arterial pressure wave expanding or contracting a blood vessel underlying the tissue in the position. A processing component is configured to input the signal and compute from the signal pulse wave parameters representing detected characteristics of the progressing arterial pressure wave of the user. The processing component configured to compute from the pulse wave parameters blood pressure value of the user.

According to one embodiment a method is presented, comprising monitoring blood pressure information of a user with a device, comprising a pressure sensor, and a fastening element. The method comprises the steps of detachably attaching the pressure sensor to a position on the outer surface of a tissue of a user, generating with the pressure sensor a signal that varies according to deformations of the tissue in response to an arterial pressure wave expanding or contracting a blood vessel underlying the tissue in the position. The method further comprises inputting by a processing component the signal and computing from the signal pulse wave parameters representing detected characteristics of the progressing arterial pressure wave of the user and computing by the processing component from the pulse wave parameters blood pressure value of the user.

### BRIEF DESCRIPTION OF THE FIGURES

In the following the invention will be described in greater detail, in connection with preferred embodiments, with reference to the attached drawings, in which

FIGS. 1a and 1b illustrate functional elements of example embodiments of a device;

FIGS. 2a and 2b illustrates functional example configuration of a blood pressure information monitoring system;

FIGS. 3a and 3b illustrate example arrangements of sensors in the device;

FIG. 4 illustrates an example pulse wave;

FIG. 5 illustrates an example flow chart of a measurement;

FIG. 6 illustrates an example chart of parameters and coefficients.

### DETAILED DESCRIPTION OF SOME EMBODIMENTS

The following embodiments are exemplary. Although the specification may refer to “an”, “one”, or “some” embodiment(s), this does not necessarily mean that each such reference is to the same embodiment(s), or that the feature only applies to a single embodiment. Single features of different embodiments may be combined to provide further embodiments.

In the following, features of the invention will be described with a simple example of a device architecture in which various embodiments of the invention may be implemented. Only elements relevant for illustrating the embodiments are described in detail. Various implementations of blood measurement devices and blood pressure information monitoring systems comprise elements that are generally known to a person skilled in the art and may not be specifically described herein.

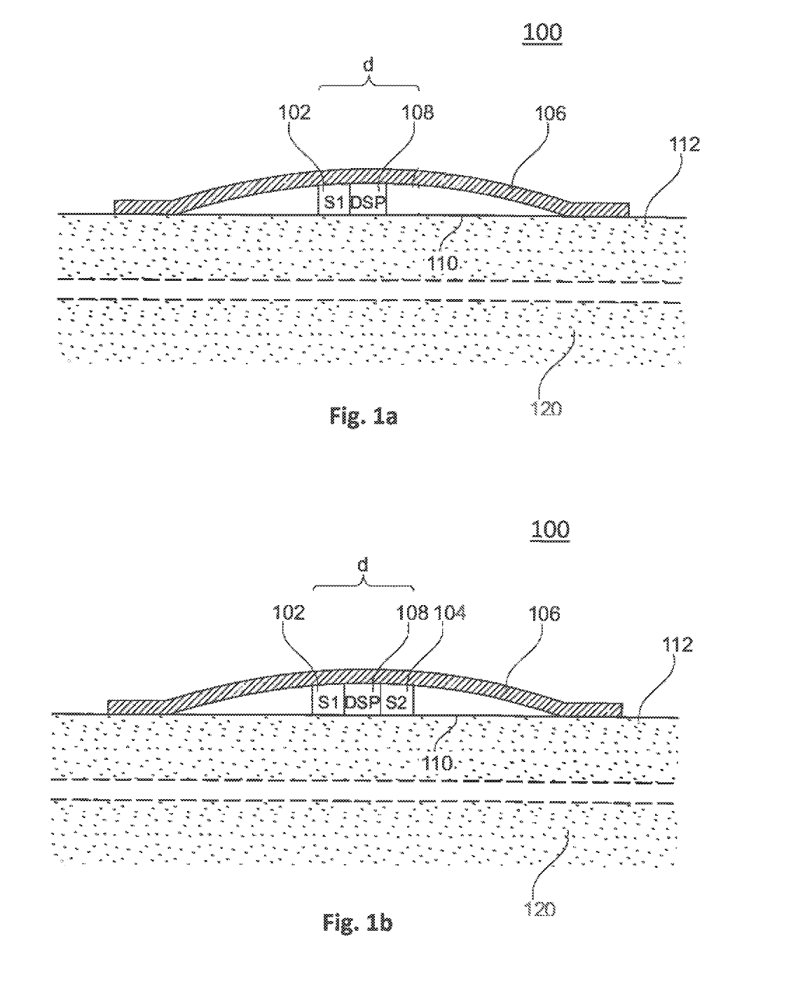

The monitoring system according to the invention comprises a device that generates one or more output values that represent detected characteristics of arterial pressure waves of a user. These values may be used as such or be further processed to indicate blood pressure information of the user. The block charts of FIGS. 1a and 1b illustrates functional elements of embodiments of a device 100 according to examples of the present invention. It is noted that the Figure is schematic; some proportions of the elements may be exaggerated to demonstrate the functional concepts of the embodiment. The device 100 comprises a first pressure sensor (S1) 102, an optional second pressure sensor (S2) 104, a fastening element 106, and a processing component (DSP) 108. It is noted that in some embodiments the device 100 may comprise more than two pressure sensors.

A pressure sensor refers here to a functional element that converts ambient pressure into mechanical displacement of a diaphragm, and translates the displacement into an electrical signal. It is noted that the device 100 comprises at least one pressure sensor. It is clear to a person skilled in the art that additional pressure sensors may be included to the device without deviating from the scope of protection. Any pressure sensor of the pressure sensors included in a device may be applied in the claimed manner. Advantageously capacitive high resolution pressure sensors are applied due to their low power consumption and excellent noise performance. Other types of pressure sensors, for example piezoresistive pressure sensors, may be applied, however, without deviating from the scope of protection. The first pressure sensor 102 is detachably attached to a first position, and the optional second pressure sensor 104 is detachably attached to a second position on the outer surface 110 of a tissue 112 of a user. The first position and the second position are separated by a predefined sensor distance d. The positions are selected such that the sensors are placed along a blood vessel 120 underneath the tissue of the user. The positions may be, for example, in an arm of a user. Other positions on the body of the user may be applied as well within the scope of protection. The tissue 112 may be for example skin of the user.

The at least one pressure sensor is attached to the tissue with a fastening element 106 such that when an arterial pressure wave of blood expands or contracts the blood vessel 120 underlying the tissue, the tissue deforms and the pressure between the tissue and the fastening element varies according to deformations of the tissue. The fastening element 106 refers here to mechanical means that may be applied to position the pressure sensors 102, 104 into contact with the outer surface 110 of the tissue 112 of the user. The fastening element 106 may be implemented, for example, with an elastic or adjustable strap. The pressure sensors 102, 104 and any electrical wiring required by their electrical connections may be attached or integrated to one surface of at least part of the strap. Other mechanisms may be applied, and fastening element 106 may apply other means of attachment, as well. For example, fastening element 106 may comprise easily removable adhesive bands to attach the pressure sensors on the tissue.

The device also comprises a processing component 108 that is electrically connected to the first pressure sensor 102 and the second pressure sensor 104 for further processing input signals generated by the pressure sensors. The processing component 108 illustrates here any configuration of processing elements included in the device 100. Advanced microelectromechanical pressure sensors are typically packaged sensor devices that include a micromachined pressure sensor and a measuring circuit. In addition, the device 100 may include a further processing element into which pre-processed signals from the pressure sensor are delivered through predefined sensor device interfaces.

The processing component 108 may be a combination of one or more computing devices for performing systematic execution of operations upon predefined data. Such processing component essentially comprises one or more arithmetic logic units, a number of special registers and control circuits. The processing component 108 may comprise or may be connected to a memory unit that provides a data medium where computer-readable data or programs, or user data can be stored. The memory unit may comprise volatile or non-volatile memory, for example EEPROM, ROM, PROM, RAM, DRAM, SRAM, firmware, programmable logic, etc.

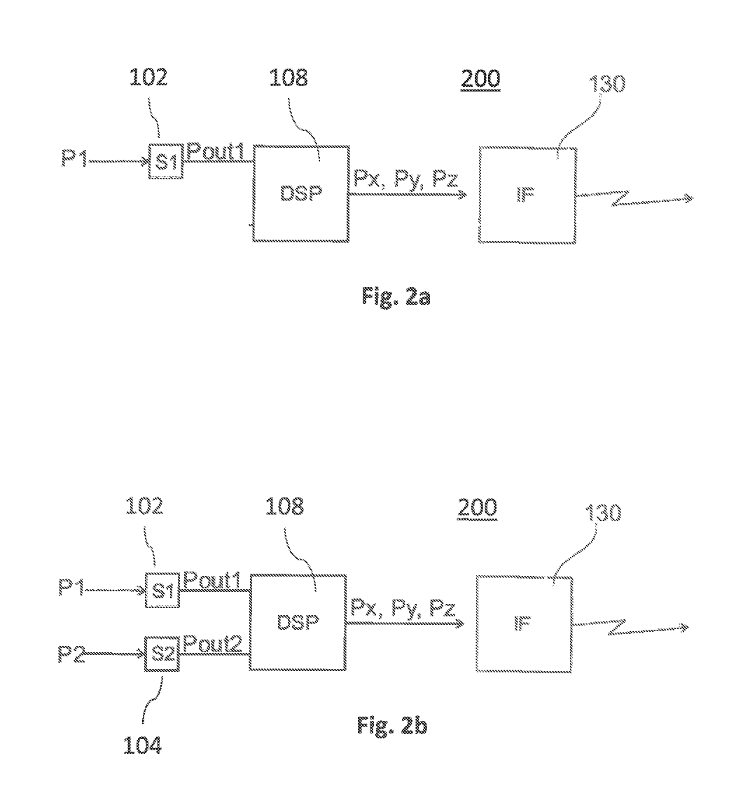

FIGS. 2a and 2b illustrate a functional configuration of a blood pressure information monitoring system 200 that includes the device 100 of FIG. 1. Accordingly, the first pressure sensor 102 in the first position is exposed to pressure P1, and is configured to generate a first signal Pout1. The first signal corresponds to a pressure between the fastening element 106 and the tissue 112 of the user, which pressure varies according to deformations of the tissue 112 when an arterial pressure wave expands or contracts a blood vessel 120 underneath the tissue 112 in the first position. Correspondingly, the optional second pressure sensor 104 is exposed to pressure P2, and is configured to generate a second signal Pout2. The second signal corresponds to a pressure between the fastening element 106 and the tissue 112 of the user, which pressure varies according to deformations of the tissue in response to the arterial pressure wave expanding or contracting the blood vessel 120 underlying the tissue in the second position.

The first signal Pout1 and the optional second signal Pout2 are input to the processing component 108 that is configured to use them to compute one or more output values Px, Py, Pz, each of which represents a detected characteristic of the arterial pressure wave of the user. The detected characteristic may be, for example, a detected pressure exerted by the arterial pressure wave upon the walls of the underlying blood vessel, a speed of propagation of the arterial pressure wave, or shape of the waveform of the arterial pressure wave. These output values may be utilised output as such to the user through a user interface included in or integrated with the device, or they may be delivered to an external server component for further processing.

The device 100 may thus comprise, or be connected to an interface unit 130 that comprises at least one input unit for inputting data to the internal processes of the device, and at least one output unit for outputting data from the internal processes of the device.

If a line interface is applied, the interface unit 130 typically comprises plug-in units acting as a gateway for information delivered to its external connection points and for information fed to the lines connected to its external connection points. If a radio interface is applied, the interface unit 130 typically comprises a radio transceiver unit, which includes a transmitter and a receiver. The transmitter of the radio transceiver unit receives a bit stream from the processing component 108, and converts it to a radio signal for transmission by the antenna. Correspondingly, the radio signals received by the antenna are led to the receiver of the radio transceiver unit, which converts the radio signal into a bit stream that is forwarded for further processing to the processing component 108. Different radio interfaces may be implemented with one radio transceiver unit, or separate radio transceiver units may be provided for the different radio interfaces.

The interface unit 130 may also comprise a user interface with a keypad, a touch screen, a microphone, and equals for inputting data and a screen, a touch screen, a loudspeaker, and equals for outputting data.

The processing component 108 and the interface unit 130 are electrically interconnected to provide means for performing systematic execution of operations on the received and/or stored data according to predefined, essentially programmed processes. These operations comprise the procedures described for the device and the blood pressure information monitoring system.

The monitoring system may also comprise a remote node (not shown) communicatively connected to the device 100 attached to the user. The remote node may be an application server that provides a blood pressure monitoring application as a service to a plurality of users. Alternatively, the remote node may be a personal computing device into which a blood pressure monitoring application has been installed.

While various aspects of the invention may be illustrated and described as block diagrams, message flow diagrams, flow charts and logic flow diagrams, or using some other pictorial representation, it is well understood that the illustrated units, blocks, apparatus, system elements, procedures and methods may be implemented in, for example, hardware, software, firmware, special purpose circuits or logic, a computing device or some combination thereof. Software routines, which are also called as program products, are articles of manufacture and can be stored in any apparatus-readable data storage medium and they include program instructions to perform particular predefined tasks. The exemplary embodiments of this invention also provide a computer program product, readable by a computer and encoding instructions for monitoring blood pressure information of a user in a device of FIGS. 1a or 1b or a system of FIG. 2a or 2b.

Also other characteristics of the arterial pressure wave may be measured for further blood pressure information. For example, it is easily understood that the first signal and the optional second signal have a similar waveform. One may select a reference point from the waveform (e.g. maximum, minimum) and detect occurrence of this reference point in the first signal and in the second signal. A time interval between an instance of the reference point in the waveform of the first signal and an instance of the reference point in the waveform of the second signal corresponds to the time needed by the pressure wave to progress from the first pressure sensor to the second pressure sensor. It is thus possible to compute a speed of propagation of the arterial pressure wave of the user by dividing the predefined sensor distance by the determined time interval. It is known that the speed of the blood pressure wave in a blood vessel may be used to indicate stiffness of the walls of the blood vessel.

As another aspect, also the shape of the waveform may be used to indicate stiffness of the walls of the blood vessel. For example, it is known that a reflection wave seen close to the peak typically indicates increased stiffness in the blood vessel. It is possible to measure this estimated stiffness by computing from a waveform a value (e.g. the height of the pulse vs. the width of the pulse) and use that to indicate the interesting stiffness characteristic of the arterial pressure wave.

An important enabling factor for this novel solution has been the high resolution achieved with the advanced capacitive pressure sensors. As an example, the noise given in a data sheet of a pressure sensor component SCP1000 of Murata Electronics is 1.5 Pa@1.8 Hz and 25 μA. This corresponds to a noise density of 1.1 Pa/√Hz, which is equivalent to 0.11 mm blood assuming a density of 1 kg/l. If the predefined sensor distance is, for example, 1 cm and the gain factor is 1, a one second measurement gives a calibration error of the order of 1% (standard deviation). This is well adequate for non-invasive blood pressure measurements.

The proposed solution provides a user-friendly, stress-minimizing and still accurate method for measuring and monitoring blood pressure information. The configuration is inherently robust, because positioning of the pressure sensors in respect of the artery is not as sensitive to errors as adjusting the elements in the conventional optical arrangements. In addition, calibration of the device is quick and easy, and can be implemented without measurements with additional reference equipment.

As discussed earlier, the detected characteristic may be, for example, the detected pressure exerted by the arterial pressure wave upon the walls of the underlying blood vessel. Any measurement arrangement, however, is dependent on the measurement arrangements and conditions. In order to have comparable reference values, the output values need to be calibrated. In the present configuration, calibration is simple and can be performed without additional measurement devices.

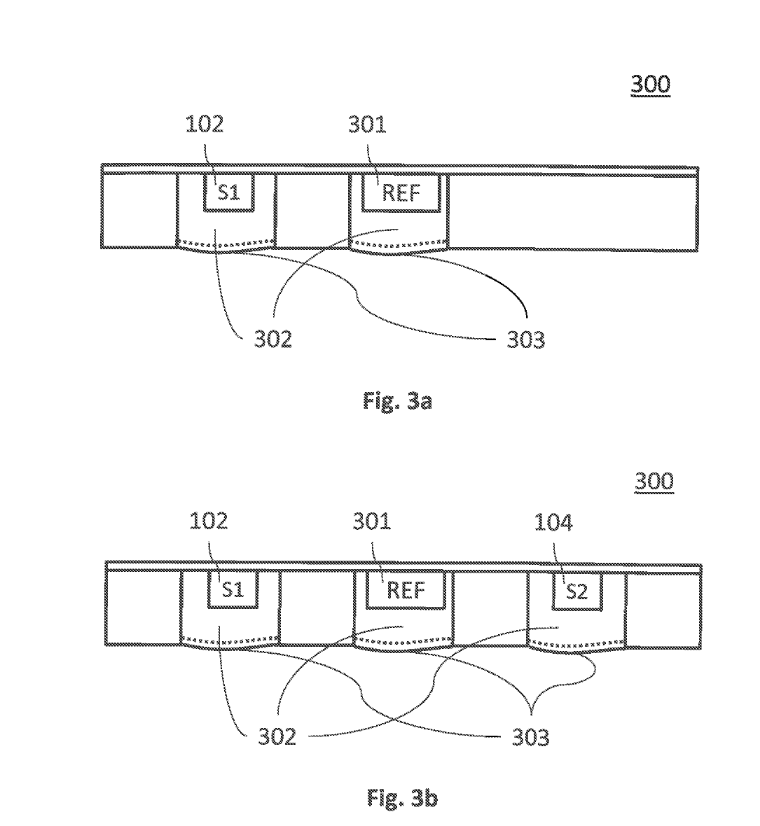

FIGS. 3a and 3b illustrate example embodiments of a sensor arrangement 300. An optional reference capacitor 301 (REF) is located between the first pressure sensor 102 and the optional second pressure sensor 104. The pressure sensors 102, 104 and the reference capacitor 301 may be situated in cavities 302 arranged in the sensor arrangement. Looking from below the cavities may be for example cylindrical, cubical or any other suitable form. The cavities 302 may be filled with a substance like gel etc. to achieve a liquid contact between the tissue and the pressure sensors 102, 104 and the reference capacitor 301 to efficiently convey pulsation. A diaphragm 303 may be arranged to cover the cavity 302. It is to be noted that the sensor arrangement 300 is an example and the number and locations of the pressure sensors and the reference capacitor 301 may vary. There may be more than one pressure sensor 102, 104 and/or reference capacitor in a same cavity 302.

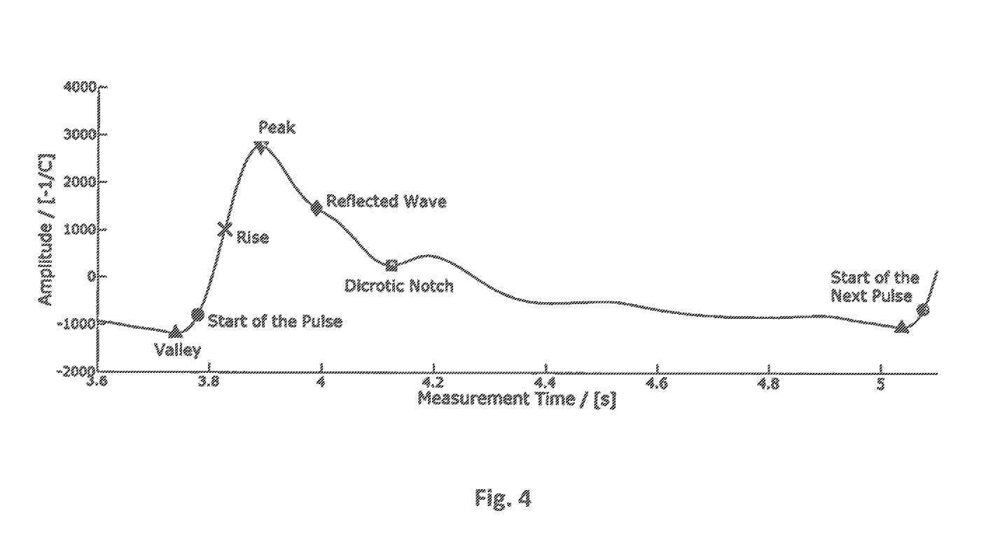

FIG. 4 illustrates an example pulse wave with few example points which may be used for blood pressure monitoring. The blood pressure is the pressure the blood exerts against the walls of a blood vessel. The pulse wave, or the pulse pressure wave, is the result of the propagation of pressure wave, not blood itself, in the blood vessels. In the cardiac cycle, it is highest during ventricular contraction, or systole, and lowest during ventricular relaxation, or diastole. Systolic blood pressure (SBP) refers to the highest aortic pressure in ventricular contraction, and diastolic blood pressure (DBP) to the lowest aortic pressure after ventricular relaxation and before the opening of the aortic valve. Blood pressure may be reported as millimeter of mercury (mmHg); 1 mmHg equals to about 133,32 pascals. A typical SBP is 120 mmHg and DBP 80 mmHg, or 120/80 mmHg.

The example points in the example pulse wave of FIG. 4 are explained next. It is to be noted that these points are exemplary only. Number of points can be identified and analysed using common signal analysis methods. The absolute values of the points may be used in the monitoring as well as their relative positions on the pulse wave.

Valley: indicates a position in the pulse wave where the measured pressure is at the lowest, diastolic valley.

Rise: indicates a position in the pulse wave where the measured pressure is rising. It may be the point where the rising is fastest.

Peak: indicates a position in the pulse wave where the measured pressure is at the highest, systolic peak

Reflected wave: The reflected wave is caused by a discontinuity in blood vessels, for example, when larger arteries divide to smaller ones. The discontinuity occurs in multiple sites in the circulatory system, such as in high-resistance arterioles in abdomen, and each site creates a reflected wave which combine to form a single wave.

Dicrotic notch: is the result of the closure of the aortic valve. The pressure is higher after the dicrotic notch due to capacitive behavior of the aorta: right before the closure of the aortic valve, the blood momentarily flows back to the heart thus lowering the pressure in the aorta; next, as the pressure is lower, the aorta releases stored mechanical energy and pushes blood forward. This creates a pressure wave that amplifies the primary pulse wave.

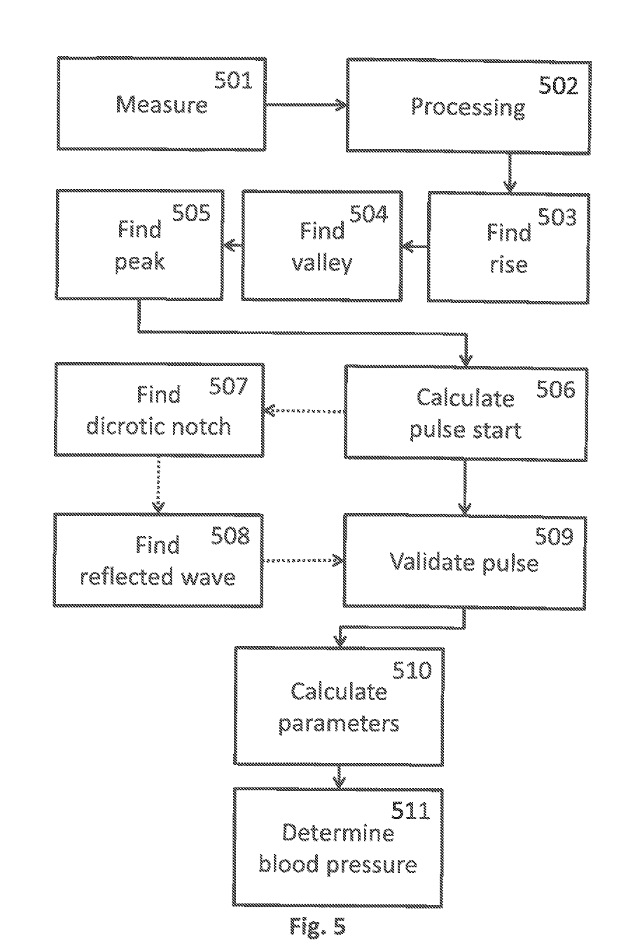

FIG. 5 illustrates an example flow chart of a pulse wave measurement process for identifying e.g. the example points illustrated in FIG. 4. Receiving pulse wave data 501 from the pressure sensors 102, 104 may include short term or continuous monitoring. The measurement may be real-time or the received pulse wave data may first be stored somewhere and analysed later. The measured pulse wave data may be processed using common signal processing means. The processing 502 may include high-pass filtering for example with a cut-off frequency of 0.1 Hz. The processing 502 may include low pass filtering for example with a cut-off frequency of 30 Hz. For example exponential weighted averaging filter, a spike filter, S-G filtering (Savitzky-Golay) or other suitable filtering methods with near-linear phase shift retaining the original shape of the pulse may be used. The processing 502 may also include differentiating the pulse wave data for example once or twice. Between all differentiations the signal can be S-G filtered to minimize noise.

Analysing the pulse wave data may include finding a rise 503, finding a valley 504 and finding a peak 505. Based on these findings a start of a pulse can be calculated 506. The pulse wave may be analysed further to find a dicrotic notch 507 and a reflected wave 508. After finding the rise, the peak, the valley and possibly the dicrotic notch and the reflected wave, the pulse may be validated 509 and further desired parameters calculated 510. Using at least the parameters a value for blood pressure can be determined 511.

After validating a pulse from the measured pulse wave data and when the measured pulse wave data has been analysed, certain pulse wave parameters can be calculated using the detected characteristics. These pulse wave parameters may include: heart rate or beat-to-beat time, pulse wave velocity, time to systolic peak, time to reflected wave, relative height of the reflected wave, time to dicrotic notch, and relative height of the dicrotic notch etc. Beat-to-beat time can be calculated for example as the time between consecutive rises. The pulse wave velocity can be calculated using the distance between radial and brachial measurement location divided by the time difference between for example rises in the signals. Relative heights can be calculated as a difference between the amplitude of a point and valley in relation to difference between peak and valley.

For the determination of blood pressure, mean values of the pulse wave parameters can be used. In addition to pulse wave parameters used in the determination of blood pressure, other aspects of the pulse can be also measured. These include ensemble averaging of pulses, heart rate variability and pulse pressure variability, standard deviation of heart rate variability and rough estimation of cardiac output.

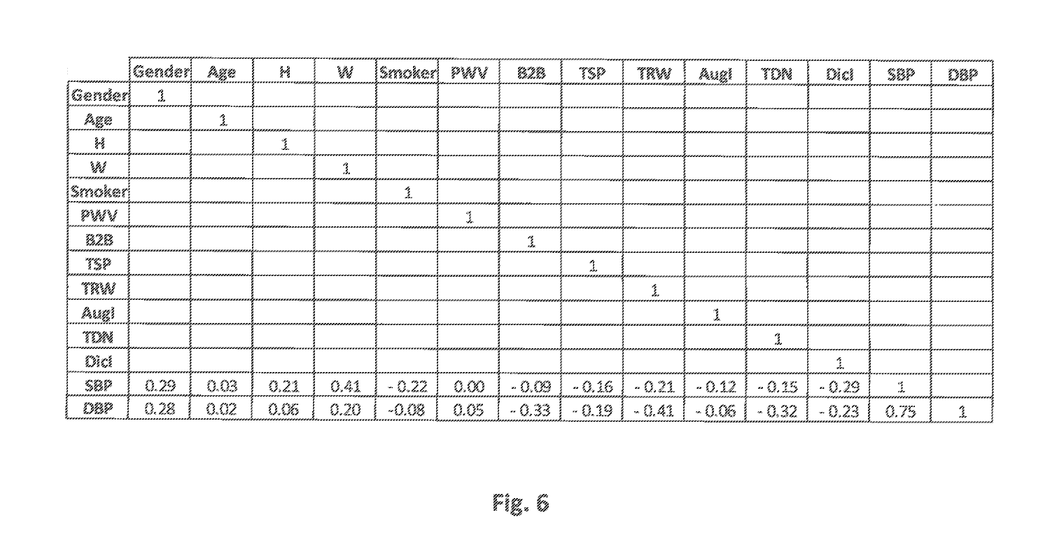

In addition to the pulse wave parameters certain user related parameters may be used. FIG. 6 illustrates example parameters and corresponding correlation coefficients for pulse wave parameters and person related parameters. The pulse wave parameters and the user related parameters are used to determine the blood pressure of the user.

The user related parameters may include user's sex, height (H), weight (W), age, habits like smoking (Not-S) etc. The pulse wave parameters may include pulse wave velocity (PWV), beat to beat (B2B), time to systolic peak (TSP), time to reflective wave (TRW), relative amplitude of the reflective wave (AugI), time to dicrotic notch (TDN), relative amplitude of the dicrotic notch (Did). The pulse wave velocity (PWV) can be calculated using pulse transit time (PTT) from one pressure sensor to another and the distance between the two pressure sensors.

Using the parameters and corresponding correlation coefficients, equations for estimating systolic blood pressure SBP and diastolic blood pressure DBP can be created. Example equations are presented below, where URP is the user related parameters combined for simplicity. The PRP can be calculated for example:

Fᵤᵣₚ=g₁*gender+h₁*height+w₁*weight+s₁*smoker

### SYSTOLIC BLOOD PRESSURE, SBP

F_{sbp}=URP+sp₁*TSP+rw₁*TRW+a₁*AugI+dn₁*TDN+d₁*DicI

Where, sp₁, rw₁, a₁, dn₁, and d₁ are coefficients for corresponding measured parameters.

### DIASTOLIC BLOOD PRESSURE, DBP

F_{dbp}=URP+b₂* B2B+sp₂*TSP+rw₂*TRW+dn₂*TDN+d₂*DicI

Where, b₂, sp₂, rw₂, dn_{2 and} d₂ are coefficients for corresponding measured parameters.

The results for both SBP and DBP can be made more accurate, if the equations are e.g. of power two or three. For all equations presented, the coefficients can be optimized by calculating least mean squares between estimation of a blood pressure and reference measurements. Other suitable optimization methods may be used too.

SBP=k_{sbp;1}*F_{sbp}+k_{sbp,2}*F_{sbp}²+k_{sbp,3}*F_{sbp}³

DBP=k_{dbp,1}*F_{dbp}+k_{dbp,2}*F_{dbp}²+k_{dbp,3}*F_{dbp}³

One advance of the current invention is that there is no need to measure absolute blood pressure values. Using the relative values of the parameters and correlative coefficients values representing blood pressure can be determined.

It is apparent to a person skilled in the art that as technology advances, the basic idea of the invention can be implemented in various ways. The invention and its embodiments are therefore not restricted to the above examples, but they may vary within the scope of the claims

1. A device for evaluating blood pressure, comprising:
at least one pressure sensor;
a fastening element for detachably attaching the pressure sensor to a position on the outer surface of a tissue of a user; wherein
the pressure sensor is configured to generate a signal that varies according to deformations of the tissue in response to an arterial pressure wave expanding or contracting a blood vessel underlying the tissue in the position;
a processing component configured to input the signal and compute from the signal pulse wave parameters representing detected characteristics of the progressing arterial pressure wave of the user; and
the processing component configured to compute from the pulse wave parameters a blood pressure value of the user.

2. The device of claim 1, wherein the blood pressure value comprises at least one of the following: diastolic blood pressure or systolic blood pressure.

3. The device of claim 1, wherein the processing component is configured to compute an output value representing the waveform of the signal.

4. The device of claim 1, wherein the processing component is configured to use relative values of the pulse wave parameters to compute blood pressure value of the user.

5. The device of claim 1, further comprising:
a second pressure sensor;
the fastening element for detachably attaching the second pressure sensor to a second position on the outer surface of a tissue of a user; wherein
the second pressure sensor is configured to generate a second signal that varies according to deformations of the tissue in response to an arterial pressure wave expanding or contracting a blood vessel underlying the tissue in the second position; and wherein
the processing component configured to input the second signal and compute from the second signal pulse wave parameters representing detected characteristics of the progressing arterial pressure wave of the user.

6. The device of claim 5, wherein the processing component is configured to compute from the signals from the at least one pressure sensor and the second pressure sensor a velocity for a pulse wave.

7. A blood pressure monitoring system, comprising a device according to claim 1.

8. A method, comprising:
monitoring blood pressure information of a user with a device, comprising a pressure sensor, and a fastening element;
detachably attaching the pressure sensor to a position on the outer surface of a tissue of a user;
generating with the pressure sensor a signal that varies according to deformations of the tissue in response to an arterial pressure wave expanding or contracting a blood vessel underlying the tissue in the position;
inputting by a processing component the signal and computing from the signal pulse wave parameters representing detected characteristics of the progressing arterial pressure wave of the user; and
computing by the processing component from the pulse wave parameters blood pressure value of the user.

9. The method of claim 8, wherein
the blood pressure value comprises at least one of the following: diastolic blood pressure or systolic blood pressure.

10. The method of claim 8, further comprising computing an output value representing the shape of the waveform of the signal.

11. The method of claim 8, further comprising using relative values of the pulse wave parameters to compute blood pressure value of the user.

12. The method of claim 8, further comprising:
monitoring blood pressure information of a user with a device, comprising a second pressure sensor;
detachably attaching the second pressure sensor to a second position on the outer surface of a tissue of a user;
generating with the second pressure sensor a second signal that varies according to deformations of the tissue in response to an arterial pressure wave expanding or contracting a blood vessel underlying the tissue in the second position; and
inputting by the processing component the second signal and compute from the second signal pulse wave parameters representing detected characteristics of the progressing arterial pressure wave of the user.

13. The method of claim 5, further comprising computing from the signals from the at least one pressure sensor and the second pressure sensor a velocity for a pulse wave.

14. A computer program product embodied on a non-transitory computer-readable medium, and encoding instructions for executing a method of claim 7 in a blood pressure monitoring system.
